(19) United States
(12) Patent Application Publication
(10) Pub. No.: US 2024/0358466 A1
(43) Pub. Date: Oct. 31, 2024

(54) Surgical Cart With Robotic Arm

(71) Applicant: Medtronic Navigation, Inc., Louisville, CO (US)

(72) Inventor: Richard Todd Hage, Castle Rock, CO (US)

(21) Appl. No.: 18/608,505

(22) Filed: Mar. 18, 2024

Related U.S. Application Data
(60) Provisional application No. 63/462,094, filed on Apr. 26, 2023.

(51) Int. Cl. A61B 50/26; A61B 34/00; A61B 34/20; A61B 34/30

(52) U.S. Cl. CPC A61B 50/26; A61B 34/20; A61B 34/25; A61B 34/30

(57) ABSTRACT

A surgical system including a base assembly having an actuator vertically movable along a z-axis. A motor in cooperation with the actuator to vertically move the actuator along the z-axis. A support member mounted to the actuator and configured to support a robotic arm. The support member movable by the actuator to vertically move the robotic arm along the z-axis. A linear sensing device mounted to the base assembly. The linear sensing device configured to connect to an operating table and sense vertical movement of the operating table relative to the base assembly. A control module configured to receive sensor signals from the linear sensing device identifying a vertical position of the operating table, and configured to operate the motor to vertically move the actuator, the support member, and the robotic arm when mounted to the support member in unison with the operating table.

### CROSS-REFERENCE TO RELATED APPLICATION

This application claims the benefit of and priority to U.S. Provisional Patent Application No. 63/462,094 filed Apr. 26, 2023, the entire disclosure of which is incorporated by reference herein.

### FIELD

The subject disclosure is directed to a surgical cart including a robotic arm having a z-axis adjustment.

### BACKGROUND

This section provides background information related to the present disclosure, which is not necessarily prior art.

Robotic arms are used during operating room procedures to perform a variety of different tasks. For example, a robotic arm may be used to maneuver a surgical instrument and to assist with imaging. The robotic arm may be mounted to the operating table or on a mobile cart. While current robotic arms are suitable for their intended use, they are subject to improvement. The present disclosure includes a surgical system including an improved robotic arm that provides numerous advantages, such as those set forth herein.

### SUMMARY

This section provides a general summary of the disclosure, and is not a comprehensive disclosure of its full scope or all of its features.

The present disclosure includes a surgical system including a base assembly having an actuator vertically movable along a z-axis. A motor in cooperation with the actuator to vertically move the actuator along the z-axis. A support member mounted to the actuator and configured to support a robotic arm. The support member movable by the actuator to vertically move the robotic arm along the z-axis. A linear sensing device mounted to the base assembly. The linear sensing device configured to connect to an operating table and sense vertical movement of the operating table relative to the base assembly. A control module configured to receive sensor signals from the linear sensing device identifying a vertical position of the operating table, and configured to operate the motor to vertically move the actuator, the support member, and the robotic arm when mounted to the support member in unison with the operating table.

The present disclosure further includes a surgical system with a surgical tracking system defining a navigation space having a navigation coordinate system. A robotic arm has a robotic coordinate system. A base assembly includes an actuator. The robotic arm is supported by the actuator. The actuator is vertically movable between a first position and a second position to vertically move the robotic arm along a z-axis. A linear sensing device is mounted to the base assembly. The linear sensing device is configured to connect to an operating table and sense vertical movement of the operating table relative to the base assembly. A base assembly control module is configured to receive sensor signals from the linear sensing device identifying a vertical position of the operating table, and configured to operate the motor to vertically move the actuator, the support member, and the robotic arm in unison with the operating table. A navigation processor system is configured to register the robotic coordinate system to the navigation space. Operation of the motor by the base assembly control module to vertically move the robotic arm in unison with the operating table maintains registration of the robotic coordinate system to the navigation space throughout vertical movement of both the robotic arm and the operating table.

The present disclosure also provides for a method of operating a surgical system. The method includes the following: positioning a robotic assembly relative to an operating table, the robotic assembly including a robotic arm, an actuator configured to vertically raise and lower the robotic arm, a motor configured to actuate the actuator, a sensing device configured to sense vertical movement of the operating table, and a control module in communication with the sensing device and the motor; connecting the robotic assembly to the operating table such that vertical movement of the operating table is sensed by the sensing device; and registering, with a navigation processor system, a robotic coordinate system of the robotic arm to a navigation space of a surgical tracking system. The control module of the mobile cart is configured to receive sensor signals from the linear sensing device identifying a vertical position of the operating table, and configured to operate the motor to vertically move the robotic arm in unison with the operating table.

### DRAWINGS

The drawings described herein are for illustrative purposes only of selected embodiments and not all possible implementations, and are not intended to limit the scope of the present disclosure.

FIG. 1 is diagrammatic view illustrating an overview of a surgical system in accordance with the present disclosure including a mobile cart with a robotic arm;

FIG. 2A illustrates an operating table and the robotic arm of FIG. 1 both in a lowered position;

FIG. 2B illustrates the operating table and the robotic arm of FIG. 1 both in a raised position;

FIG. 3 illustrates a method in accordance with the present disclosure for registering the robotic arm to a navigation space of the surgical system; and

FIG. 4 illustrates a method in accordance with the present disclosure for using the mobile cart to maintain relative height of the robotic arm to an operating room table as the table is raised and lowered along the z-axis.

Corresponding reference numerals indicate corresponding parts throughout the several views of the drawings.

### DETAILED DESCRIPTION

Example embodiments will now be described more fully with reference to the accompanying drawings.

The subject disclosure is directed to an exemplary embodiment of a surgical procedure on a subject, such as a human patient. It is understood, however, that the system and methods described herein are merely exemplary and not intended to limit the scope of the claims included herein. In various embodiments, it is understood, that the systems and methods may be incorporated into and/or used on non-animate objects.

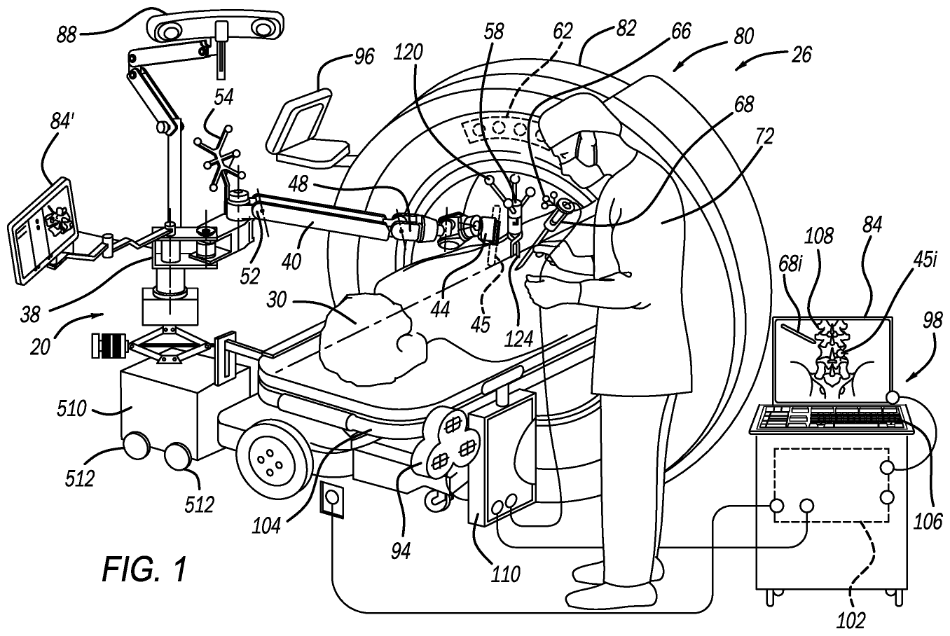

FIG. 1 is a diagrammatic view illustrating an overview of a procedure room or arena. In various embodiments, the procedure room may include a surgical suite in which may be placed a robotic system 20 and a navigation system 26 that can be used for various procedures. The robotic system 20 may include a Mazor X™ robotic guidance system, sold by Medtronic, Inc. The robotic system 20 may be used to assist in guiding selected instrument, such as drills, screws, etc. relative to a subject 30. As discussed herein, the robotic system 20 may be registered or correlated to a selected coordinate system such as via image-to image registration or correlation or similar methods, such as disclosed in U.S. Pat. No. 11,135,025, incorporated herein by reference. The robotic system 20 will be described further herein.

The robotic system 20 may include one or more arms 40 that are moveable or pivotable relative to the subject 30, such as including an end effector 44 and mounted, such as moveably mounted to the base 38. The end effector 44 may be any appropriate portion, such as a tube, guide, or passage member. The end effector 44 may be moved relative to the base 38 with one or more motors. The position of the end effector 44 may be known or determined relative to the base 38 with one or more encoders at one or more joints, such as a wrist joint 48 and/or an elbow joint 52 of the robotic system 20.

The robotic system 20 is arranged relative to an operating room table 104. The operating room table 104 may be any suitable operating table configured to be laterally moved in the X-Y axis, and vertically raised/lowered in the Z-axis. Exemplary tables 104 are described further herein. The robotic system 20, particularly the base 38 and arm 40, are also movable in the z-axis, as explained further herein. Based on user preference and the type of procedure to be performed, the height of the base 38 and the arm 40 are vertically set in the Z-axis relative to the table 104 by the operator at an operating position. Once the operating position is set, it is desirable to maintain the operating position throughout the procedure. Thus, if the table 104 is raised or lowered, it is desirable to raise or lower the base 38 and arm 40 the same distance along the Z-axis to maintain the relative position between the table 104 and the base 30 and the arm 40 at the relative operating position. As described herein, the present disclosure includes a system for maintaining this relative position.

The navigation system 26 can be used to track the location of one or more tracking devices. The tracking devices may include a robot tracking device 54, a subject tracking device 58, an imaging system tracking device 62, and/or an tool tracking device 66. A tool 68 may be any appropriate tool such as a drill, forceps, or other tool operated by a user 72. The tool 68 may also include an implant, such as a spinal implant or orthopedic implant. It should further be noted that the navigation system 26 may be used to navigate any type of instrument, implant, or delivery system, including: guide wires, arthroscopic systems, orthopedic implants, spinal implants, deep brain stimulation (DBS) probes, etc. Moreover, the instruments may be used to navigate or map any region of the body. The navigation system 26 and the various instruments may be used in any appropriate procedure, such as one that is generally minimally invasive or an open procedure.

An imaging device 80 may be used to acquire pre-, intra-, or post-operative or real-time image data of a subject, such as the subject 30. It will be understood, however, that any appropriate subject can be imaged and any appropriate procedure may be performed relative to the subject. In the example shown, the imaging device 80 comprises an O-arm® imaging device sold by Medtronic Navigation, Inc. having a place of business in Louisville, Colorado, USA. The imaging device 80 may have a generally annular gantry housing 82 in which an image capturing portion is moveably placed. The image capturing portion may include an x-ray source or emission portion and an x-ray receiving or image receiving portion located generally or as practically possible 180 degrees from each other and mounted on a rotor relative to a track or rail. The image capturing portion can be operable to rotate 360 degrees during image acquisition. The image capturing portion may rotate around a central point or axis, allowing image data of the subject 30 to be acquired from multiple directions or in multiple planes. The imaging device 80 can include those disclosed in U.S. Pat. Nos. 7,188,998; 7,108,421; 7,106,825; 7,001,045; and 6,940,941; all of which are incorporated herein by reference, or any appropriate portions thereof. In one example, the imaging device 80 can utilize flat plate technology having a 1,720 by 1,024 pixel viewing area. As is understood by on skilled in the art and mentioned herein, the imaging system 80 may be any appropriate imaging system such as the O-arm imaging system, a C-arm imaging system, etc.

The imaging device 80 can also be tracked with the tracking device 62. The image data defining an image space acquired of the patient 30 can, according to various embodiments, be inherently or automatically registered relative to an object space. The object space can be the space defined by a patient 30 in the navigation system 26. The automatic registration can be achieved by including the tracking device 62 on the imaging device 80 and/or the determinable precise location of the image capturing portion. According to various embodiments, as discussed herein, imageable portions, virtual fiducial points and other features can also be used to allow for registration, automatic or otherwise. It will be understood, however, that image data can be acquired of any subject which will define subject space. Patient space is an exemplary subject space. Registration allows for a translation between patient space and image space.

The patient 80 can also be tracked as the patient moves with a patient tracking device, DRF, or the tracker 58. Alternatively, or in addition thereto, the patient 30 may be fixed within navigation space defined by the navigation system 26 to allow for registration. As discussed further herein, registration of the image space to the patient space or subject space allows for navigation of the instrument 68 with the image data. When navigating the instrument 68, a position of the instrument 68 can be illustrated relative to image data acquired of the patient 30 on a display device 84. Various tracking systems, such as one including an optical localizer 88 or an electromagnetic (EM) localizer 92 can be used to track the instrument 68.

It is further appreciated that the imaging device 80 may be an imaging device other than the O-arm® imaging device and may include in addition or alternatively a fluoroscopic C-arm. Other exemplary imaging devices may include fluoroscopes such as bi-plane fluoroscopic systems, ceiling mounted fluoroscopic systems, cath-lab fluoroscopic systems, fixed C-arm fluoroscopic systems, isocentric C-arm fluoroscopic systems, 3D fluoroscopic systems, etc. Other appropriate imaging devices can also include MRI, CT, ultrasound, etc.

In various embodiments, an imaging device controller 96 may control the imaging device 80 and can receive the image data generated at the image capturing portion and store the images for later use. The controller 96 can also control the rotation of the image capturing portion of the imaging device 80. It will be understood that the controller 96 need not be integral with the gantry housing 82, but may be separate therefrom. For example, the controller may be a portions of the navigation system 26 that may include a processing and/or control system 98 including a processing unit or processing portion 102. The controller 96, however, may be integral with the gantry 82 and may include a second and separate processor, such as that in a portable computer.

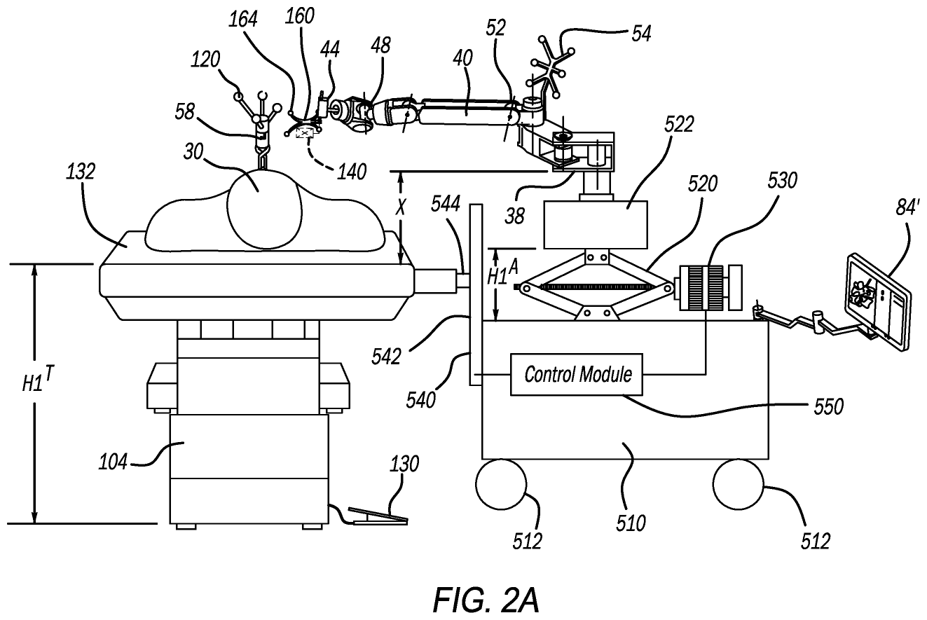

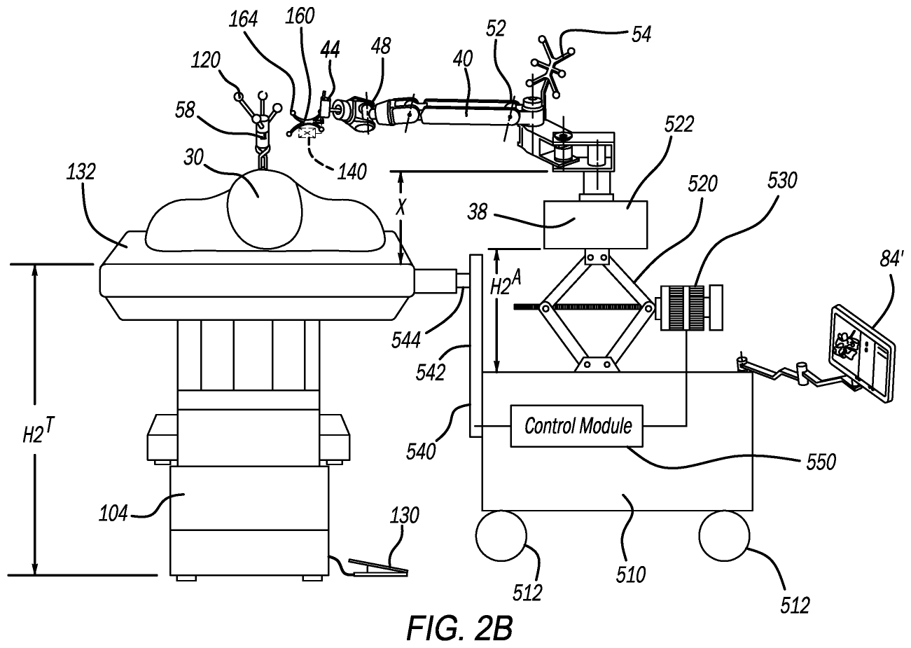

The patient 30 can be fixed onto an operating table 104. The operating table 104 may be any suitable operating table configured to be raised and lowered along a z-axis by an operator. For example, the operating table 104 may be raised or lowered using foot pedals 130 of FIGS. 2A and 2B. According to one example, the table 104 can be an Axis Jackson® operating table sold by OSI, a subsidiary of Mizuho Ikakogyo Co., Ltd., having a place of business in Tokyo, Japan or Mizuho Orthopedic Systems, Inc. having a place of business in California, USA. Patient positioning devices can be used with the table, and include a Mayfield® clamp or those set forth in commonly assigned U.S. patent application Ser. No. 10/405,068 entitled “An Integrated Electromagnetic Navigation And Patient Positioning Device”, filed Apr. 1, 2003 which is hereby incorporated by reference.

The position of the patient 30 relative to the imaging device 80 can be determined by the navigation system 26. The tracking device 62 can be used to track and locate at least a portion of the imaging device 80, for example the gantry or housing 82. The patient 30 can be tracked with the dynamic reference frame 58. Accordingly, the position of the patient 30 relative to the imaging device 80 can be determined. Further, the location of the imaging portion can be determined relative to the housing 82 due to its precise position on the rail within the housing 82, substantially inflexible rotor, etc. The imaging device 80 can include an accuracy of within 10 microns, for example, if the imaging device 80 is an O-Arm® imaging device sold by Medtronic Navigation, Inc. having a place of business in Louisville, Colorado. Precise positioning of the imaging portion is further described in U.S. Pat. Nos. 7,188,998; 7,108,421; 7,106,825; 7,001,045; and 6,940,941; all of which are incorporated herein by reference.

According to various embodiments, the imaging device 80 can generate and/or emit x-rays from the x-ray source that propagate through the patient 30 and are received by the x-ray imaging receiving portion. The image capturing portion generates image data representing the intensities of the received x-rays. Typically, the image capturing portion can include an image intensifier that first converts the x-rays to visible light and a camera (e.g. a charge couple device) that converts the visible light into digital image data. The image capturing portion may also be a digital device that converts x-rays directly to digital image data for forming images, thus potentially avoiding distortion introduced by first converting to visible light.

Two dimensional and/or three dimensional fluoroscopic image data that may be taken by the imaging device 80 can be captured and stored in the imaging device controller 96. Multiple image data taken by the imaging device 80 may also be captured and assembled to provide a larger view or image of a whole region of a patient 30, as opposed to being directed to only a portion of a region of the patient 30. For example, multiple image data of the patient's 30 spine may be appended together to provide a full view or complete set of image data of the spine.

The image data can then be forwarded from the image device controller 96 to the navigation computer and/or processor system 102 that can be a part of a controller or work station 98 having the display 84 and a user interface 106. It will also be understood that the image data is not necessarily first retained in the controller 96, but may also be directly transmitted to the work station 98. The work station 98 can provide facilities for displaying the image data as an image 108 on the display 84, saving, digitally manipulating, or printing a hard copy image of the received image data. The user interface 106, which may be a keyboard, mouse, touch pen, touch screen or other suitable device, allows the user 72 to provide inputs to control the imaging device 80, via the image device controller 96, or adjust the display settings of the display 84. The work station 98 may also direct the image device controller 96 to adjust the image capturing portion of the imaging device 80 to obtain various two-dimensional images along different planes in order to generate representative two-dimensional and three-dimensional image data.

With continuing reference to FIG. 1, the navigation system 26 can further include the tracking system including either or both of the electromagnetic (EM) localizer 94 and/or the optical localizer 88. The tracking systems may include a controller and interface portion 110. The controller 110 can be connected to the processor portion 102, which can include a processor included within a computer. The EM tracking system may include the STEALTHSTATION® AXIEM™ Navigation System, sold by Medtronic Navigation, Inc. having a place of business in Louisville, Colorado; or can be the EM tracking system described in U.S. patent application Ser. No. 10/941,782, filed Sep. 15, 2004, and entitled “METHOD AND APPARATUS FOR SURGICAL NAVIGATION”; U.S. Pat. No. 5,913,820, entitled “Position Location System,” issued Jun. 22, 1999; and U.S. Pat. No. 5,592,939, entitled “Method and System for Navigating a Catheter Probe,” issued Jan. 14, 1997; all of which are herein incorporated by reference. It will be understood that the navigation system 26 may also be or include any appropriate tracking system, including a STEALTHSTATION® TREON® or S7™ tracking systems having an optical localizer, that may be used as the optical localizer 88, and sold by Medtronic Navigation, Inc. of Louisville, Colorado. Other tracking systems include an acoustic, radiation, radar, etc. The tracking systems can be used according to generally known or described techniques in the above incorporated references. Details will not be included herein except when to clarify selected operation of the subject disclosure.

Wired or physical connections can interconnect the tracking systems, imaging device 80, etc. Alternatively, various portions, such as the instrument 68 may employ a wireless communications channel, such as that disclosed in U.S. Pat. No. 6,474,341, entitled “Surgical Communication Power System,” issued Nov. 5, 2002, herein incorporated by reference, as opposed to being coupled directly to the controller 110. Also, the tracking devices 62, 66, 54 can generate a field and/or signal that is sensed by the localizer(s) 88, 94.

Additional representative or alternative localization and tracking systems are set forth in U.S. Pat. No. 5,983,126, titled “Catheter Location System and Method,” issued Nov. 9, 1999, which is hereby incorporated by reference. The navigation system 26 may be a hybrid system that includes components from various tracking systems.

According to various embodiments, the navigation system 26 can be used to track the instrument 68 relative to the patient 30. The instrument 68 can be tracked with the tracking system, as discussed above. Image data of the patient 30, or an appropriate subject, can be used to assist the user 72 in guiding the instrument 68. The image data, however, is registered to the patient 30. The image data defines an image space that is registered to the patient space defined by the patient 30. The registration can be performed as discussed herein, automatically, manually, or combinations thereof.

Generally, registration allows a translation map to be generated of the physical location of the instrument 68 relative to the image space of the image data. The translation map allows the tracked position of the instrument 68 to be displayed on the display device 84 relative to the image data 108. A graphical representation 68i, also referred to as an icon, can be used to illustrate the location of the instrument 68 relative to the image data 108.

A registration system or method can use the tracking device 58. The tracking device 58 may include portions or members 120 that may be trackable, but may also act as or be operable as a fiducial assembly. The fiducial assembly 120 can include a clamp or other fixation portion 124 and the imageable fiducial body 120. It is understood, however, that the members 120 may be separate from the tracking device 58. The fixation portion 124 can be provided to fix any appropriate portion, such as a portion of the anatomy. The fiducial assembly 120 can be interconnected with a portion of a spine of the subject 30. The fiducial portions 120 may be imaged with the imaging device 80. It is understood, however, that various portions of the subject (such as a spinous process) may also be used as a fiducial portion.

In various embodiments, when the fiducial portions 120 are imaged with the imaging device 80, image data is generated that includes or identifies the fiducial portions 120. The fiducial portions 120 can be identified in image data automatically (e.g. with a processor executing a program), manually (e.g. by selection an identification by the user 72), or combinations thereof (e.g. by selection an identification by the user 72 of a seed point and segmentation by a processor executing a program). Methods of automatic imageable portion identification include those disclosed in U.S. Pat. No. 8,150,494 issued on Apr. 3, 2012, incorporated herein by reference. Manual identification can include selecting an element (e.g. pixel) or region in the image data wherein the imageable portion has been imaged. Regardless, the fiducial portions 120 identified in the image data can be used as fiducial points or positions that can be used to register the image data or the image space of the image data with patient space.

In various embodiments, to register an image space or coordinate system to another space or coordinate system, such as a navigation space, the fiducial portions 120 that are identified in the image 108 may then be identified in the subject space defined by the subject 30, in an appropriate manner. For example, the user 72 may move the instrument 68 relative to the subject 30 to touch the fiducial portions 120, if the fiducial portions are attached to the subject 30 in the same position during the acquisition of the image data to generate the image 108. It is understood that the fiducial portions 120, as discussed above in various embodiments, may be attached to the subject 30 and/or may include anatomical portions of the subject 30. Additionally, a tracking device may be incorporated into the fiducial portions 120 and they may be maintained with the subject 30 after the image is acquired. In this case, the registration or the identification of the fiducial portions 120 in a subject space may be made. Nevertheless, according to various embodiments, the user 72 may move the instrument 68 to touch the fiducial portions 120. The tracking system, such as with the optical localizer 88, may track the position of the instrument 68 due to the tracking device 66 attached thereto. This allows the user 72 to identify in the navigation space the locations of the fiducial portions 120 that are identified in the image 108. After identifying the positions of the fiducial portions 120 in the navigation space, which may include a subject space, the translation map may be made between the subject space defined by the subject 30 in a navigation space and the image space defined by the image 108. Accordingly, identical or known locations allow for registration as discussed further herein.

During registration, a translation map is determined between the image data coordinate system of the image data such as the image 108 and the patient space defined by the patient 30. Once the registration occurs, the instrument 68 can be tracked with the tracking system that is registered to the image data to allow an identification and illustration of a position of the tracked instrument 68 as an icon superimposed on the image data. Registration of the image 108 (or any selected image data) to the subject 30 may occur at any appropriate time.

After the registration of the image space to the patient space, the instrument 68 can be tracked relative to the image 108. As illustrated in FIG. 1, the icon 68i representing a position (which may include a 6 degree of freedom position (including 3D location and orientation)) of the instrument 68 can be displayed relative to the image 108 on the display 84. Due to the registration of the image space to the patient space, the position of the icon 68i relative to the image 108 can substantially identify or mimic the location of the instrument 68 relative to the patient 30 in the patient space. As discussed above, this can allow a navigated procedure to occur.

The robotic system 20 having the robotic system coordinate system may be registered to the navigation space coordinate system, as discussed herein, due to the reference tracking device 54 (e.g. if fixed to a known position on or relative to the robotic system 20) and/or due to the tracking of the snapshot tracking device 160 (FIGS. 2A and 2B). The snapshot tracking device 160 may include one or more trackable portions 164 that may be tracked with the localizer 88 or any appropriate localizer (e.g. optical, EM, radar). It is understood, however, that any appropriate tracking system may be used to track the snapshot tracking device 160. A fixed reference tracking device may also be positioned within the navigation space. The fixed navigation tracker may include the patient tracker 58, which may be connected to the patient 30 and/or the robot tracker 54 that may be fixed to the base 34 of the robotic system 20. The reference tracker, therefore, may be any appropriate tracker that is positioned relative to the snapshot tracker 160 that is within the navigation coordinate space during the registration period. For the discussion herein, the robot tracker 54 will be referred to however, the patient tracker 58 may also be used as the reference tracker. Further, reference tracker may be positioned within the coordinate system at any position relative to the snapshot tracker 160 as long as the snapshot tracker 160 may be tracked relative to the reference tracker.

In various embodiments, the snapshot tracker 160 may be positioned at a known position relative to the end effector 44. For example, the snapshot tracker 160, as illustrated in FIGS. 2A and 2B, which includes the trackable portions 164, extends from a rod or connection member. The localizer 88 may then view or determine a position of the snapshot tracking device 160 relative to the reference tracking device 54 and or the reference tracking device 58. As the localizer 88 defines or may be used to define the navigation space, determining or tracking a position of the snapshot tracking device 160 relative to the reference frame 54 may be used to determine a relationship between a position within the navigation space and the robotic space of the end effector 44.

The navigation space defined by the localizer 88 may include the full navigation space, which may include portions relative to the subject, such as the subject tracker 58 and other portions that may be moved therein, such as the instrument 68. The robotic registration space may be smaller and may include a robotic registration space that may include the reference frame 54 and the snapshot tracker 160. As discussed above, however, the robot registration navigation space may include the snapshot tracker 160 and the patient tracker 58 for registration. Accordingly, the exemplary registration navigation space is merely for the current discussion. As discussed herein, both the robotic reference tracker 54 and the patient tracker 58 need not be used simultaneously. This is particularly true when the patient 30 is fixed in space, such as fixed relative to the robotic system 20.

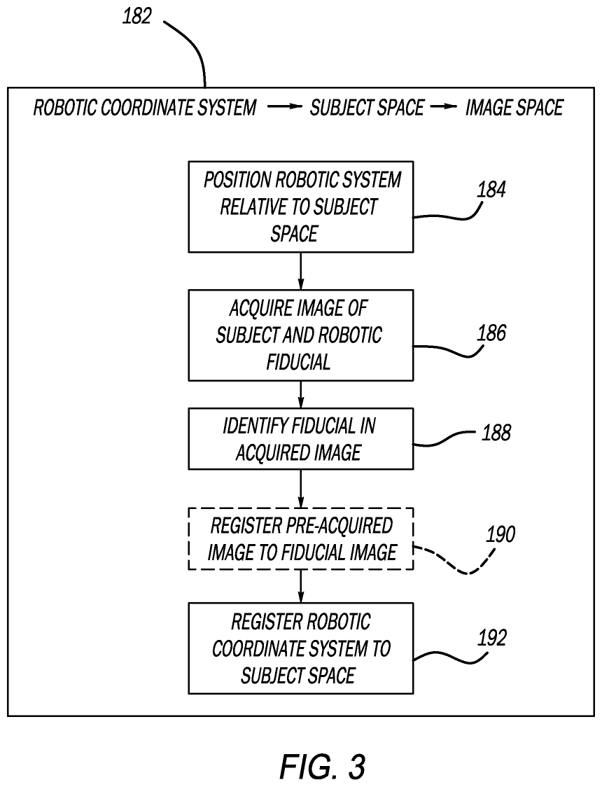

With additional reference to FIG. 3, a process or method of coordinating or registering a robotic coordinate system of the robotic system 20 to a navigation space or navigation coordinate system of the navigation system 26 is described. The registration may include various portions or sub-parts, as discussed herein. The various parts may occur in any appropriate order, and the order discussed herein is merely exemplary. The co-registration may further allow only one coordinate system of the robotic or navigation to be registered to a third coordinate system, such as an image coordinate system, but allow the registration of the other to the third coordinate system.

The robotic system 20, as discussed above, is positioned relative to the subject 30 for various portions of a procedure. In various embodiments, the robotic system 20 may be registered to the subject 30 and to the image 108 of the subject 30, that may be displayed on the display device 84 and/or a second or auxiliary display device 84′ that may be movable relative to the robotic system 20. The imaging system 80, or any appropriate imaging system, may be used to image the subject 30. The image may include a portion of the subject, such as one or more of the vertebrae and a fiducial or robotic fiducial array 140 (FIGS. 2A, 2B) that may be fixed to the robotic system 20. The robotic fiducial 140 may be fixed to a selected portion of the robotic system 20, such as to the base 34 and/or the fixed portion 38. The robotic fiducial 140 may also and/or alternatively be connected to the end effector 44 (illustrated in phantom in FIGS. 2A and 2B). The robotic fiducial 140 may be positioned relative to the subject 30 for acquisition of images such that the fiducial 140 is apparent in the images. Upon acquisition of the image of the robotic fiducial 140 and portions of the subject 30, such as the vertebrae, the position of the robotic fiducial 140 relative to the vertebrae may be determined. If the robotic fiducial 140 is fixed to the robotic system 20, the robotic coordinate system may be determined relative to the subject space coordinate system. In various embodiments, if the fiducial 140 is connected to the end effector 44, the known position of the end effector in the robotic coordinate system allows for image registration to the robotic coordinate system of the robotic system 20. The robotic coordinate system may be registered to a subject space or coordinate system in the method 182 of FIG. 3.

Generally, the registration may include positioning the robotic system 20 relative to a subject space in block 184. Positioning of the robotic system 20 relative to the subject space may include positioning the robotic system 30 relative to the subject. Further, positioning of the robotic system 20 may include positioning or removably positioning the robotic fiducial 140 relative to the subject 30. The robotic fiducial 140 may be removably placed in a position relative to the robotic system 20 for various procedures and may be substantially positioned in the same position for different or subsequent procedures. With the subject 30 positioned relative to the robotic system 20, fiducial images may be acquired of the subject 30 and the robotic fiducial 140 with the imaging system 80 in block 186. The acquisition of the fiducial images in block 186 allows for image data to be acquired of the subject 30, such as with the vertebrae, and the fiducial 140. It is desirable to maintain a relative position along the Z-axis between an upper surface 132 of the table 104 and a base 38 of the robotic arm 40 throughout an operating procedure. The relative position may be set based on operator preference, as well as based on the procedure being performed. Once the relative position is set, the relative position is maintained as the table 104 is raised or lowered along the Z-axis by sensing movement of the table 104, and then simultaneously (or nearly simultaneously) moving the base 38 along the Z-axis in the same direction as the table 104 to maintain the relative operating position as the table 104 is moved.

The processor 102 and/or the controller 96 is configured to save the position of the robotic arm 40 (including the Euclidian positions and orientations of the fiducial 140 and the robotic arm 40, including the end effector 44) at the same time that the image was acquired in block 186 so that the processor 102 and/or the controller 96 know the precise location of the robotic system 20 during registration of the robotic coordinate system to the subject space described at block 192.

As discussed above, the image acquired of the fiducial 140 may be used for registration of a coordinate reference frame of the robotic arm 20 and the pre-acquired image, which may be any appropriate type of image. The pre-acquired image may be a two- and/or three-dimensional image (such as a CT image, MRI image, or the like). The image acquired of the fiducial 140 may also be any appropriate type of image, such as a two-dimensional fluoroscopic image, a two-dimensional image acquired with the O-arm® imaging system, a cone beam image required with the O-arm® imaging system, or other appropriate image data.

After acquisition of the robotic fiducial image in block 186, identifying of the robotic fiducial 140 in the acquired fiducial images occurs in block 188. Identification of the robotic fiducial in the robotic fiducial images may be manual, automatic, or a combination of automatic and manual. For example, the user may identify the robotic fiducial in the image a selected automatic system may segment the fiducials from the fiducial images, or the user may identify a seed pixel or voxel or multiple seed pixels or voxels and the processor system may further segment the fiducial system.

In various embodiments, the acquired images in block 186 may be used for planning and/or performing a procedure. For example, the imaging system 80 may acquire image data sufficient for a selected procedure. Thus, the images acquired in block 186 may be used for planning and navigating a selected procedure relative to the subject 30. The image data may include two-dimensional image data, reconstructed three-dimensional image data, and/or image data acquired over time to illustrate movement of motion of the subject (which may be acquired in 2D or 3D).

In various embodiments, however, the fiducial image acquired in block 186 may be optionally registered to other-time or pre-acquired images in block 190, such as an MRI or a computed tomography scan of the subject 30 prior to the acquisition of the fiducial images in block 186. The pre-acquired images may be acquired at any appropriate time prior to the acquisition of the fiducial images in block 186. It is understood, however, that the images may be acquired after the fiducial images and may be registered to the fiducial images in a similar manner as discussed herein. The registration of the fiducial images to the pre-acquired images may occur in any appropriate manner such as segmentation of selected vertebrae, identification in registration of selected fiducial elements in the images (e.g. anatomical fiducial portions and/or positioned or implanted fiducial members) or other appropriate procedures. Generally, the Mazor X® Robotic System may generally allow for registration of a pre-acquired image to the fiducial images and may be appropriate for registering the fiducial images in block 186 to the pre-acquired images in the registration of the pre-acquired image to the fiducial image in block 190.

The robotic coordinate system may also be registered to the subject space in block 192 with the identification of fiducials in the image in block 188 and the registration. The robotic fiducial 140, imaged with the fiducial images in block 186, is positioned in a known position relative to the robotic system 20, such as the base 34 and/or with the known position of the end effector 44 in the robotic coordinate system. The robotic coordinate system that is defined by the robotic system 20 relative to the base 34 and/or the fixed portion 38 may, therefore also, be pre-determined or known relative to the robotic fiducial 140 as the robotic fiducial 140 is fixed relative to the robotic system 20. When positioned with the end effector 44, the position of the robotic fiducial 140 is known in the robotic coordinate system by tracked (e.g. robotic system tracking) movement of the end effector 44. The fiducial image acquired in block 186 may also assist in defining the patient space relative to which the robotic system 20, particularly the end effector movable portion 44, may move is also then known. As discussed above, the end effector 44 moves in the robotic coordinate system due to the robotic tracking system that may include various mechanisms, such as encoders at the various movable portions, such as the wrist 48 or elbow 52, of the robotic system 20. If the fiducial images in block 186 are the images for performing the procedure, such as for navigation and may the displayed image 108, the registration may be substantially automatic as the subject 30 may be substantially fixed relative to the robotic system 20 (e.g. with a fixation ember extending from the base 38) and connected to the subject 30, such as the vertebrae 126.

Accordingly the robotic coordinate system can be registered to the subject space and/or image space according to the method 182. Given the registration of the robotic coordinate system to the image space the robotic, coordinate system registration may be used to determine a position of the end effector 44 and/or a member positioned through or with the end effector 44, relative to the image 108. Accordingly, the image 108 may be used to display a graphical representation, such as a graphical representation of the member or instrument 45 as an icon 45i superimposed or superimposed relative to the image 108.

Therefore, the registration or translation of the robotic system 20 and its respective coordinate system relative to the subject 30 may be made, as discussed above and in U.S. Pat. No. 11,135,025 titled “System and method for Registration Between Coordinate Systems and Navigation,” which is assigned to Medtronic Navigation, Inc. of Louisville, CO and is incorporated herein by reference in its entirety. The fiducial image may acquire an image of the subject 30 and the fiducial 140 at substantially the same time and include portions of the subject 30 and the fiducial 140 in the same image or image data. The fiducial image that includes an image of a portion of the fiducial 140 and the subject 30 may be used to register or generate a translation map between the fiducial image and any pre-acquired image, such as of the subject 30. The translation map between the fiducial image strand of the pre-acquired image may allow for registration between the two images.

Due to the registration between the two images, the pose of the fiducial 140 may be determined relative to a pre-acquired image. This may allow for a pose of the robotic system 20 to be determined relative to the pre-required image. Thus, any planning that may have occurred in the pre-acquired image may be translated or registered to the current pose of the robotic system 20 relative to the subject 30. The fiducial image that is inclusive of the image of the fiducial 140 and the subject 30 may be, therefore, registered to the pre-acquired image for us during a procedure using the robotic system 20 and relative to the subject 30.

The robotic system 20 advantageously maintains registration between the robotic coordinate system and the subject space/navigation coordinate system even if the surgical table is raised or lowered after registration. With reference to FIGS. 1, 2A, and 2B, the robotic system 20 includes a base assembly 510, which may be configured as a mobile cart including wheels 512. Alternatively, the base assembly 510 may be a stationary base, which may be arranged in an operating room, for example. For exemplary purposes only, the base assembly 510 is referred to herein as the cart or mobile cart.

The mobile cart 510 includes an actuator 520 mounted thereto, which is movable between a lowered position as illustrated in FIG. 2A, and a raised position as illustrated in FIG. 2B. The actuator 520 may be stopped at any intermediate position as well. The actuator 520 may be any suitable actuator, such as a scissor jack, a telescoping actuator, screw drive, etc. A support member 522 is mounted to the actuator 520 and configured to support the robotic arm 40. The robotic arm 40 may be mounted to the support member 522 in any suitable manner.

A motor 530 is in cooperation with the actuator 520, and configured to vertically move the actuator 520 along the z-axis between the lowered position of FIG. 2A and the raised position of FIG. 2B, and to any intermediate position between the lowered position and the raised position. The motor 530 may be any suitable motor, such as a stepper motor.

The mobile cart 510 includes a linear sensing device 540. The linear sensing device 540 is configured to be connected to any suitable operating room table, such as the table 104. When connected to the table 104, the linear sensing device 540 is configured to sense when the table 104 is raised and lowered, and the distance that the table 104 is raised and lowered. The linear sensing device 540 may be any suitable device configured to identify vertical movement or z-axis movement of the table 104. For example, the linear sensing device 540 may define a track 542 that extends vertically. In cooperation with the track 542 is a link, tab or post 544, which is configured to be coupled to the table 104. The post 544 moves vertically within the track 542 as the table 104 moves up and down when the post 544 is coupled to the table 104. The vertical movement of the post 544 within the track 540 is sensed by the linear sensing device 540 in any suitable manner, such as with a linear variable displacement transducer (LVDT), or an optical sensing device. The linear sensing device 540 is in cooperation with a control module 550 of the mobile cart 510 to send signals to the control module 550 identifying the height of the table 104. The control module 550 is configured to operate the motor 530 for raising and lowing the actuator 520 based on the signals from the linear sensing device 540, as explained herein.

The mobile cart 510 further includes any suitable user interface, such as the display 84′ configured as a touch screen. The user interface may be configured in any other suitable manner as well, such as with buttons, knobs, a keyboard, etc. The user interface 84′ provides controls for the user to operate the robotic arm 40, as well as set the vertical height thereof. More specifically, the user sets the height of the robotic arm 40 relative to the table 104 by inputting the desired height into the user interface. The base 38 of the arm 40 may be set at any suitable distance X (see FIGS. 2A, 2B) along the Z-axis relative to the upper surface 132 of the table 104. The distance X may be 0, for example, such that the base 38 is aligned with the upper surface 132 at the same vertical height along the Z-axis. The control module 550 is configured to then operate the motor 530 to raise or lower the actuator 520 to the requested height relative to the table 104 to set the base 38 at the distance X relative to the upper surface 132 of the table 104.

The control module 550 is configured to receive sensor signals from the linear sensing device 540 identifying the vertical position of the operating table 104. In response to receipt of a signal that the table 104 is being raised, the control module 550 is configured to operate the motor 530 to raise the actuator 520 and the robotic arm 540 in unison with the operating table 104 to the same relative height. For example and as illustrated in FIGS. 2A and 2B, the robotic arm 40 may be set by the operator such that the base 38 of the arm 40 is at a distance X above an upper surface 132 of the table 104. As the table 104 is raised from the position H1^{T} of FIG. 2A to the position H2^{T} of FIG. 2B, the control module 550 is configured to operate the motor 530 to raise the actuator 520 from the position H1^{A} of FIG. 2A to the position H2^{A} of FIG. 2B to maintain the relative distance X of the robotic base 38 above the upper surface 132 of the table 104. Similarly, as the table 104 is lowered as detected by the linear sensor 540, the control module 550 is configured to operate the motor 530 to lower the actuator to maintain the same distance X between the base 38 of the arm 40 and the upper surface 132 of the table 104. By maintaining the distance X between the base 38 and the upper surface 132 of the table 104, the relative position of the robotic arm 40 to the navigation space will remain constant, and thus the robotic arm need not be re-registered when the table 104 is raised or lowered.

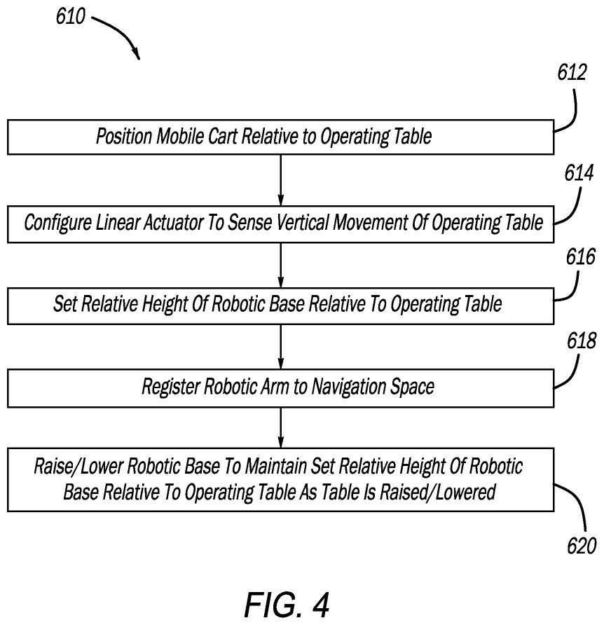

FIG. 4 illustrates an exemplary method of operating a surgical system including the mobile cart 104 in accordance with the present disclosure at reference numeral 610. At block 612, the mobile cart 104 is positioned relative to the operating table 104. At block 614, the linear sensor 540 is configured to measure vertical or z-axis movement of the OR table 104. For example, the post 544 in cooperation with the linear track 542 is coupled to the table 104 in any suitable manner. At block 616, using the user interface 84′ the height of the robotic base 38 is set relative to the table 104, such as at a first distance X relative to the upper surface 132 of the table 104. The control module 550 then operates the motor 530 to raise or lower the actuator 520 to achieve the distance X between the base 38 and the upper surface 132 of the table 104. At block 618, the robotic arm 40 is registered to the navigation space as described above. At block 620, in response to the table 104 being raised or lowered as sensed by the linear sensor 540, the control module is configured to operate the motor 540 to raise or lower the actuator 520 to maintain the height X between the robotic base 38 and the upper surface 132 of the table 104. Raising and lowering the robotic arm 40 in unison with the table 104 so that the base 38 remains at the same height at which the arm 40 was registered maintains registration of the arm 40 to the navigation system and eliminates any need for re-registration during surgery. In other words, as soon as the linear sensor 540 detects the direction of movement along the z-axis of the table 104, the control module 550 actives the motor 530 to move the actuator 520 in the same direction as the table 104 to maintain the distance X between the robotic base 38 and the upper surface 132. The movement of the actuator 520 is simultaneous with, or nearly simultaneous with, movement of the table 104 such that any difference in movement is not distinguishable by the user. Registration of the robotic arm 40 to the navigation system is advantageously maintained throughout the movement of the table 104 and the robotic arm 40.

The foregoing description of the embodiments has been provided for purposes of illustration and description. It is not intended to be exhaustive or to limit the invention. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the invention, and all such modifications are intended to be included within the scope of the invention.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

What is claimed is:

1. A surgical system comprising:
a base assembly including an actuator vertically movable along a z-axis between a first position and a second position;
a motor in cooperation with the actuator to vertically move the actuator along the z-axis;
a support member mounted to the actuator and configured to support a robotic arm, the support member movable by the actuator to vertically move the robotic arm along the z-axis;
a linear sensing device mounted to the base assembly, the linear sensing device configured to connect to an operating table and sense vertical movement of the operating table relative to the base assembly; and
a control module configured to receive sensor signals from the linear sensing device identifying a vertical position of the operating table, and configured to operate the motor to vertically move the actuator, the support member, and the robotic arm when mounted to the support member in unison with the operating table.

2. The system of claim 1, wherein the base assembly is a mobile assembly including wheels.

3. The system of claim 1, wherein the control module is included with the base assembly.

4. The system of claim 1, further comprising a user interface connected to the base assembly and in communication with the control module, the user interface configured to receive user inputs for raising and lowering the robotic arm and setting a relative position of the robotic arm relative to the operating table;
wherein the control module is configured to operate the motor to maintain the relative position between the robotic arm and the operating table as the operating table is raised and lowered.

5. The system of claim 1, wherein the actuator includes a scissor jack.

6. The system of claim 1, wherein the actuator includes a telescoping member.

7. The system of claim 1, wherein the motor includes a stepper motor.

8. The system of claim 1, further comprising the robotic arm mounted to the support member.

9. The system of claim 8, wherein the robotic arm includes an end effector configured to couple with a surgical instrument.

10. The system of claim 1, the system further comprising a processor system configured to determine a registration between a robotic coordinate system of the robotic arm and a navigation coordinate system;
wherein operation of the motor by the control module to vertically move the robotic arm in unison with the operating table maintains the registration throughout vertical movement.

11. A surgical system comprising:
a surgical tracking system defining a navigation space having a navigation coordinate system;
a robotic arm having a robotic coordinate system;
a base assembly including an actuator, the robotic arm supported by the actuator, the actuator vertically movable between a first position and a second position to vertically move the robotic arm along a z-axis;
a linear sensing device mounted to the base assembly, the linear sensing device configured to connect to an operating table and sense vertical movement of the operating table relative to the base assembly;
a base assembly control module configured to receive sensor signals from the linear sensing device identifying a vertical position of the operating table, and configured to operate the motor to vertically move the actuator, the support member, and the robotic arm in unison with the operating table; and
a navigation processor system configured to register the robotic coordinate system to the navigation space;
wherein operation of the motor by the base assembly control module to vertically move the robotic arm in unison with the operating table maintains registration of the robotic coordinate system to the navigation space throughout vertical movement of both the robotic arm and the operating table.

12. The surgical system of claim 11, wherein the base assembly is a mobile cart including wheels.

13. The surgical system of claim 12, wherein the base assembly control module is included in the cart.

14. The surgical system of claim 12, further comprising a user interface connected to the base assembly and in communication with the control module, the user interface configured to receive user inputs for raising and lowering the robotic arm and setting a relative position of the robotic arm relative to the operating table;
wherein the control module is configured to operate the motor to maintain the relative position between the robotic arm and the operating table as the operating table is raised and lowered.

15. A method of operating a surgical system, the method comprising:
positioning a robotic assembly relative to an operating table, the robotic assembly including a robotic arm, an actuator configured to vertically raise and lower the robotic arm, a motor configured to actuate the actuator, a sensing device configured to sense vertical movement of the operating table, and a control module in communication with the sensing device and the motor;
connecting the robotic assembly to the operating table such that vertical movement of the operating table is sensed by the sensing device; and
registering, with a navigation processor system, a robotic coordinate system of the robotic arm to a navigation space of a surgical tracking system;
wherein the control module of the mobile cart is configured to receive sensor signals from the linear sensing device identifying a vertical position of the operating table, and configured to operate the motor to vertically move the robotic arm in unison with the operating table.

16. The method of claim 15, further comprising:
setting, by way of a user interface of the robotic assembly, a relative position of the robotic arm relative to the operating room table;
wherein the control module is configured to operate the motor to maintain the relative position between the robotic arm and the operating table as the operating table is raised and lowered.

17. The method of claim 15, wherein the robotic assembly includes a mobile cart with wheels.

18. The method of claim 15, wherein the robotic arm includes an end effector configured to couple with a surgical instrument.
